(19) United States
(12) Patent Application Publication
(10) Pub. No.: US 2022/0183782 A1
(43) Pub. Date: Jun. 16, 2022

(54) Medical Glove Catching Device

(71) Applicant: Kellie Schweitzer, Covington, KY (US)

(72) Inventor: Kellie Schweitzer, Covington, KY (US)

(21) Appl. No.: 17/547,294

(22) Filed: Dec. 10, 2021

Related U.S. Application Data
(60) Provisional application No. 63/124,151, filed on Dec. 11, 2020.

(51) Int. Cl. A61B 42/40; B65D 83/08; B62B 5/00

(52) U.S. Cl. CPC A61B 42/40; B62B 5/00; B65D 83/0805

(57) ABSTRACT

The present invention relates generally to the field of medical gloves. More specifically, the present invention relates to a medical glove-catching device that is preferably manufactured from a rigid plastic material and has a body that is comprised of a front wall and a generally parallel rear wall with a curved lip, a bottom wall and a first side wall and generally parallel second side wall. The lip can further be placed on the bottom edge of an opening in the front surface of a COW (wherein a glove box is located) such that the device is removably-secured in a position below the opening and glove box. The body further has an interior space formed by the walls that passively catches any gloves that are unintentionally dropped when being pulled out from the glove box

### CROSS-REFERENCE TO RELATED APPLICATION

The present application claims priority to, and the benefit of, U.S. Provisional Application No. 63/124,151, which was filed on Dec. 11, 2020 and is incorporated herein by reference in its entirety.

### FIELD OF THE INVENTION

The present invention relates generally to the field of medical gloves. More specifically, the present invention relates to a medical glove catching device that is preferably manufactured from a rigid plastic material and has a body that is comprised of a front wall and a generally parallel rear wall with a curved lip, a bottom wall and a first side wall and generally parallel second side wall. The lip can further be placed on the bottom edge of an opening in the front surface of a COW (wherein a glove box is located) such that the device is removably-secured in a position below the opening and glove box. The body further has an interior space formed by the walls that passively catches any gloves that are unintentionally dropped when being pulled out from the glove box. Accordingly, the present disclosure makes specific reference thereto. Nonetheless, it is to be appreciated that aspects of the present invention are also equally applicable to other like applications, devices and methods of manufacture.

### BACKGROUND

Disposable medical gloves are used in medical environments daily to ensure a clean environment while treating patients. In many hospitals and medical facilities, boxes of clean medical gloves are stored within a computer on wheels cart (COW). However, when attempting to remove gloves from a glove box which is stored within a COW, gloves are often dropped on the floor. This is undesirable, as the glove or gloves are then no longer clean and must be disposed of. Over time, numerous gloves can be dropped in this manner, which is unnecessary and wasteful. This is especially problematic and undesirable if medical gloves are in short supply, and medical facilities cannot afford to throw multiple gloves away on a daily basis.

Therefore, there exists a long-felt need in the art for a device that prevents medical gloves from being dropped while being removed from a glove box within a COW. There also exists a long-felt need in the art for a medical glove-catching device that can be removably-attached to a COW that prevents medical gloves from being dropped while being removed from a glove box within the COW. In addition, there exists a long-felt need in the art for a medical glove catching device that can be removably-attached to a COW that prevents medical gloves from being dropped while being removed from a glove box within the COW, wherein the device is inexpensive, simple to manufacture, and can be used in a passive manner.

The subject matter disclosed and claimed herein, in one embodiment thereof, comprises a medical glove-catching device. The device is preferably manufactured from a rigid plastic material and has a body that is comprised of a front wall and a generally parallel rear wall with a curved lip, a bottom wall, and a first side wall and generally parallel second side wall. The lip can further be placed on the bottom edge of an opening in the front surface of a COW (wherein a glove box is located) such that the device is removably-secured in a position below the opening and glove box. The body further has an interior space formed by the walls that passively catches any gloves that are unintentionally dropped when being pulled out from the glove box.

In this manner, the medical glove-catching device of the present invention accomplishes all of the forgoing objectives and prevents medical gloves from being dropped on the floor and becoming unclean, such that they must be disposed of In addition, the device can be removably-attached to a COW such that the device can be used to passively catch any gloves that are unintentionally dropped. Furthermore, the device is inexpensive and simple to manufacture.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a medical glove-catching device. In the preferred embodiment, the device is comprised of a body that is further comprised of a front wall and a generally parallel rear wall with a curved lip, a bottom wall, and a first side wall and generally parallel second side wall. The body is preferably manufactured from a rigid plastic that may be transparent, semi-transparent, or opaque. The body may also be any color known in the art and may contain a plurality of indicia such as, but not limited to, patterns, logos, emblems, images, symbols, designs, letters, words, characters, animals, advertisements, brands, etc. The rear wall is further comprised of a top edge that has a curved lip that is generally shaped like an upside-down U. The lip can be placed over the bottom edge of the opening of a COW that allows a user to access a glove box within the COW. As a result, the shape of the lip removably-secures the device to the COW. However, the device can be removed from the opening by simply lifting the device upwards.

When attached to the opening of a COW, the device is positioned below the glove box stored within the COW. In this manner, any glove that is pulled from the glove box and is unintentionally dropped by a user will be caught within the interior space of the body of the device. Therefore, the glove will not fall onto the floor and will remain clean. To further ensure the device remains clean, all components of the body may have an anti-bacterial and/or anti-microbial coating.

In an additional embodiment, the rear surface of the rear wall may be comprised of at least one fastener that aids the rear wall in being secured to the front surface of the COW. In differing embodiments, the fastener may be any fastener known in the art such as, but not limited to, a magnet, a screw, a bolt, an adhesive, a hook and loop fastener, a tongue and groove fastener, etc. An additional embodiment of the device may also have at least one pocket that is adhesively or fixedly-attached to at least one wall of the body. The pocket may be manufactured from a flexible, semi-rigid or rigid material that may be any fabric or plastic known in the art and can receive items such as, but not limited to, hand sanitizer bottles or other items of the like.

Accordingly, the medical glove-catching device of the present invention is particularly advantageous as it prevents medical gloves from being dropped on the floor and becoming unclean such that they must be disposed of. In addition, the device can be removably-attached to a COW such that the device can be used to passively catch any gloves that are unintentionally dropped. Furthermore, the device is inexpensive and simple to manufacture. In this manner, the medical glove catching device provides a novel solution to the existing problem of dropping medical gloves.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front view of one potential embodiment of a medical glove-catching device of the present invention in accordance with the disclosed architecture;

FIG. 2 illustrates a perspective view of one potential embodiment of a medical glove-catching device of the present invention in accordance with the disclosed architecture; and

FIG. 3 illustrates a perspective view of one potential embodiment of a medical glove-catching device of the present invention in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a device that prevents medical gloves from being dropped while being removed from a glove box within a COW. There also exists a long-felt need in the art for a medical glove-catching device that can be removably-attached to a COW that prevents medical gloves from being dropped while being removed from a glove box within the COW. In addition, there exists a long-felt need in the art for a medical glove-catching device that can be removably-attached to a COW that prevents medical gloves from being dropped while being removed from a glove box within the COW, wherein the device is inexpensive to manufacture and can be used passively.

The present invention, in one exemplary embodiment, is comprised of a medical glove catching device. The device is comprised of a body that is further comprised of a front wall and a generally parallel rear wall with a curved lip, a bottom wall and a first side wall and generally parallel second side wall. The body is preferably manufactured from a rigid plastic that may be transparent, semi-transparent or opaque and may also be any color known in the art and contain a plurality of indicia such as, but not limited to—patterns, logos, emblems, images, symbols, designs, letters, words, characters, animals, advertisements, brands, etc. The rear wall is further comprised of a top edge that has a curved lip that is generally shaped like an upside-down U-shape. The lip can be placed over the bottom edge of the opening of a COW that allows a user to access a glove box within the COW. As a result, the shape of the lip removably-secures the device to the COW. However, the device can be removed from the opening by simply lifting the device upwards.

When attached to the opening of a COW, the device is positioned below the glove box stored within the COW. As a result, any glove that is pulled from the glove box and is unintentionally dropped by a user will be caught within the interior space of the body of the device. In this manner, the glove will not fall onto the floor and will remain clean. To further ensure the device remains clean, all components of the body may have an anti-bacterial and/or anti-microbial coating.

In an additional embodiment, the rear surface of the rear wall may be comprised of at least one fastener that aids the rear wall in being secured to the front surface of the COW. In differing embodiments, the fastener may be any fastener known in the art such as, but not limited to, a magnet, a screw, a bolt, an adhesive, a hook and loop fastener, a tongue and groove fastener, etc. An additional embodiment of the device may also have at least one pocket that is adhesively or fixedly-attached to at least one wall of the body. The pocket may be manufactured from a flexible, semi-rigid, or rigid material that may be any fabric or plastic known in the art and can receive items such as, but not limited to, hand sanitizer bottles or other items of the like.

Accordingly, the medical glove-catching device of the present invention is particularly advantageous as it prevents medical gloves from being dropped on the floor and becoming unclean, such that they must be disposed of. In addition, the device can be removably attached to a COW such that the device can be used to passively catch any gloves that are unintentionally dropped. Furthermore, the device is inexpensive and simple to manufacture. In this manner, the medical glove catching device provides a novel solution to the existing problem of dropping medical gloves.

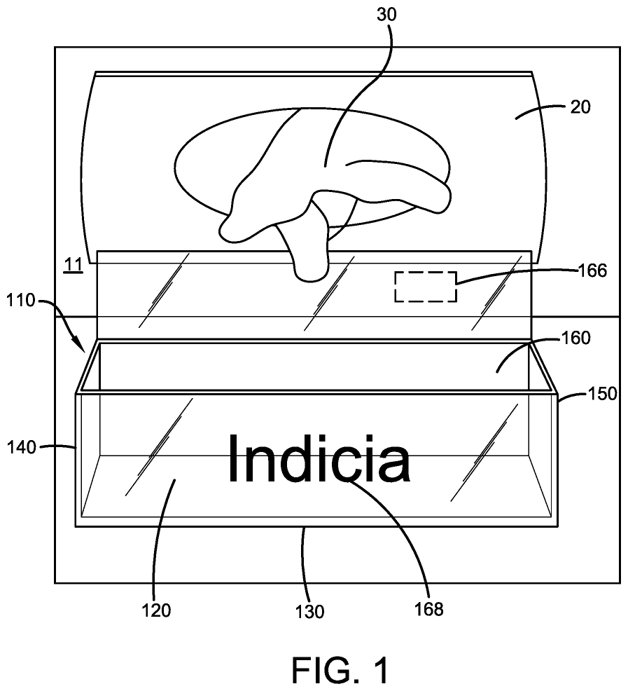

Referring initially to the drawings, FIG. 1 illustrates a front view of one potential embodiment of a medical glove-catching device 100 of the present invention in accordance with the disclosed architecture. In the preferred embodiment, the device 100 is comprised of a body 110 that is further comprised of a front wall 120 and a generally parallel rear wall 160 with a curved lip 164, a bottom wall 130, and a first side wall 140 and generally parallel second side wall 150. The body 110 is preferably generally rectangular, but may also be square, circular, triangular, etc. in differing embodiments. The body 110 is preferably manufactured from a rigid plastic such as, but not limited to, acrylic, polycarbonate, polyethylene, thermoplastic, acrylonitrile butadiene styrene, low density polyethylene, medium density polyethylene, high density polyethylene, polyethylene terephthalate, polyvinyl chloride, polystyrene, polylactic acid, acetal, nylon, fiberglass, recycled plastic, biodegradable plastic, etc., that may be transparent, semi-transparent or opaque. In addition, the body 110 may be any color known in the art and may contain a plurality of indicia 168 such as, but not limited to, patterns, logos, emblems, images, symbols, designs, letters, words, characters, animals, advertisements, brands, etc. Furthermore, the components 120, 130, 140, 150, 160 of the body 110 may be inseparable or removably-attached to one another.

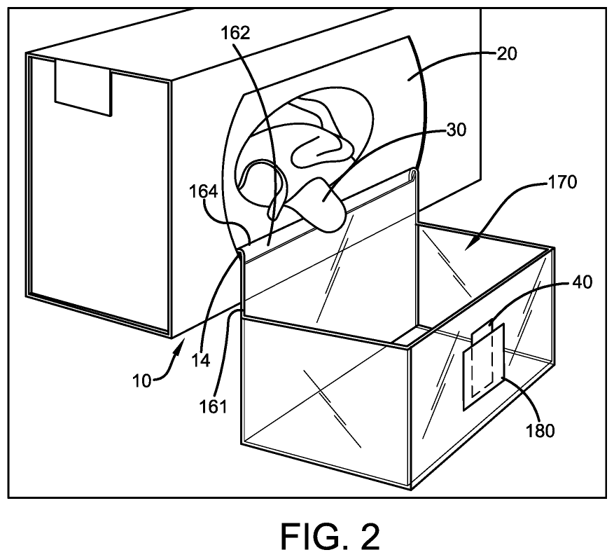

FIG. 2 illustrates a perspective view of one potential embodiment of a medical glove catching device 100 of the present invention in accordance with the disclosed architecture. The rear wall 160 is further comprised of a top edge 162 that has a curved lip 164. Said lip 164 is generally shaped like an upside-down U, and can be placed over the bottom edge 14 of the opening 12 of a COW 10. In differing embodiments, the lip 164 may also be rectangular, triangular or square-shaped. In this manner, the shape of the lip 164 removably-secures the device 100 to the opening 12 of the COW 10, wherein the device 100 can be removed from the opening 12 by simply lifting the device 100 upwards. When attached to the opening 12 of a COW 10, the device 100 is positioned below a glove box 20 that is also stored within the COW 10, wherein the box 20 can be accessed by the opening 12. In this manner, any glove 30 that is pulled from the glove box 20 and is unintentionally dropped by a user 5 will be caught within the interior space 170 of the body 110. Therefore, the glove 30 will not fall onto the floor and will remain clean. To further ensure the device 100 remains clean, all components 120, 130, 140, 150, 160 of the body 110 may have an anti-bacterial and/or anti-microbial coating.

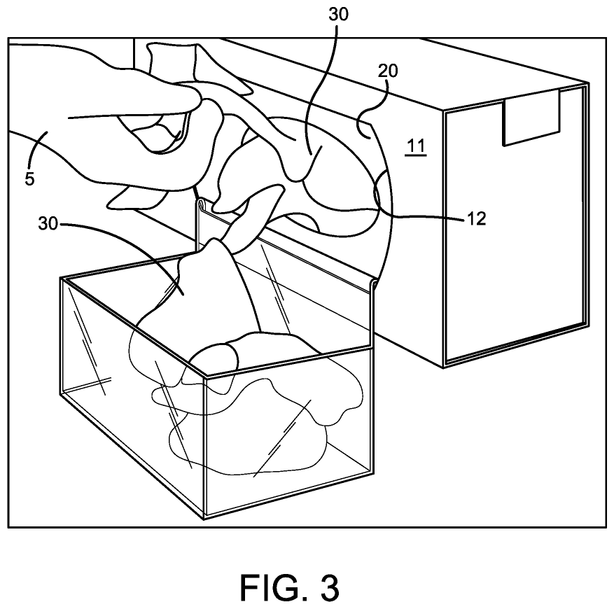

FIG. 3 illustrates a perspective view of one potential embodiment of a medical glove catching device 100 of the present invention in accordance with the disclosed architecture. In one embodiment, the rear surface 161 of the rear wall 160 may be comprised of at least one fastener 166. The fastener 166 aids the rear wall 160 in being secured to the front surface 11 of the COW 10. In differing embodiments, the fastener 166 may be any fastener known in the art such as, but not limited to, a magnet, a screw, a bolt, an adhesive, a hook and loop fastener, a tongue and groove fastener, etc. An additional embodiment of the device 100 may also have at least one pocket 180 that is adhesively or fixedly-attached to at least one of the following: the front wall 120, the first side wall 140, and/or the second side wall 150. The pocket 180 may be manufactured from a flexible, semi-rigid, or rigid material that may be any fabric or plastic known in the art. The pocket 180 can further receive items such as, but not limited to, hand sanitizer bottles 40 or other items of the like. In a further embodiment, the device 100 may be combined with the COW.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “medical glove-catching device” and “device” are interchangeable and refer to the medical glove-catching device 100 of the present invention.

Notwithstanding the forgoing, the medical glove-catching device 100 of the present invention and its various components can be of any suitable size and configuration as is known in the art without affecting the overall concept of the invention, provided that they accomplish the above-stated objectives. One of ordinary skill in the art will appreciate that the size, configuration and material of the medical glove-catching device 100 as shown in the FIGS. are for illustrative purposes only, and that many other sizes and shapes of the medical glove-catching device 100 are well within the scope of the present disclosure. Although the dimensions of the medical glove-catching device 100 are important design parameters for user convenience, the medical glove-catching device 100 may be of any size, shape and/or configuration that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A medical glove catching device comprising:
a rigid body having an interior space, wherein the rigid body is further comprised of:
a front wall;
a rear wall that is generally parallel to and spaced apart from the front wall and comprises a lip;
a bottom wall;
a first side wall; and
a second side wall that is generally parallel to and spaced apart from the first side wall.

2. The medical glove catching device of claim 1, wherein the lip is sized and configured to be placed over a bottom edge of an opening on a front surface of a computer on wheels cart in order to removably secure the medical glove catching device to the computer on wheels cart.

3. The medical glove catching device of claim 2, wherein the medical glove catching device is positioned below the opening of the front surface.

4. The medical glove catching device of claim 3, wherein a glove that is pulled from a glove box that is stored within the computer on wheels cart, and that can be pulled from the glove box via the opening on the front surface of the computer on wheels cart, is caught in the interior space of the rigid body if the glove is unintentionally dropped by a user.

5. A medical glove catching device and wheeled cart combination comprising:
a wheeled cart;
a rigid body that is generally rectangular in shape and having an interior space, wherein the rigid body is further comprised of:
a front wall and a generally parallel rear wall, wherein the generally parallel rear wall is further comprised of a curved lip;
a bottom wall; and
a first side wall and a generally parallel second side wall.

6. The medical glove catching device and wheeled cart combination of claim 5, wherein the curved lip is placed over a bottom edge of an opening on a front surface of the wheeled cart in order to removably secure the rigid body to the wheeled cart.

7. The medical glove catching device and wheeled cart combination of claim 6, wherein the rigid body is positioned below the opening of the front surface of the wheeled cart.

8. The medical glove catching device and wheeled cart combination of claim 7 further comprising at least one fastener.

9. A medical glove catching device and wheeled cart combination comprising:
a wheeled cart comprised of an opening with a bottom edge;
a rigid body having an interior space, wherein the rigid body is comprised of:
a front wall;
a rear wall having a curved lip that can be used to secure the rigid body to the bottom edge of the opening of the wheeled cart;
at least one fastener positioned along a rear surface of the rear wall;
a bottom wall;
a first side wall;
a second side wall; and
at least one pocket.

10. The medical glove catching device and wheeled cart combination of claim 9, wherein the curved lip is positioned over the bottom edge of the opening on a front surface of the wheeled cart in order to removably secure the rigid body to the wheeled cart.

11. The medical glove catching device and wheeled cart combination of claim 10, wherein the rigid body is positioned below the opening of the front surface.

12. The medical glove catching device and wheeled cart combination of claim 11, wherein a glove that is pulled from a glove box and through the opening in the wheeled cart by a user, is caught in the interior space of the rigid body if the glove is unintentionally dropped by the user.

13. The medical glove catching device and wheeled cart combination of claim 9, wherein the at least one pocket is attached to at least one of the front wall, the first side wall, or the second side wall.

14. The medical glove catching device and wheeled cart combination of claim 13, wherein the at least one pocket is rigid.

15. The medical glove catching device and wheeled cart combination of claim 10, wherein the at least one fastener is a select one of a magnet, a screw, a bolt, an adhesive, a hook and loop fastener and a tongue and grove fastener.

16. The medical glove catching device and wheeled cart combination of claim 9, wherein the rigid body comprises an anti-bacterial coating.

17. The medical glove catching device and wheeled cart combination of claim 9, wherein the rigid body is manufactured from a plastic material.

18. The medical glove catching device and wheeled cart combination of claim 9, wherein the rigid body is transparent.

19. The medical glove catching device and wheeled cart combination of claim 9, wherein the rigid body is generally rectangular.

20. The medical glove catching device and wheeled cart combination of claim 9, wherein the rigid body comprises a plurality of indicia.
